(19) United States
(12) Patent Application Publication
(10) Pub. No.: US 2026/0364114 A1
(43) Pub. Date: Nov. 19, 2026

(54) USE OF VESICLES IN PREPARATION OF DRUG FOR EXTERNAL PROCOAGULATION OR EXTERNAL HEMOSTASIS

(71) Applicant: SUN YAT-SEN UNIVERSITY, Guangzhou (CN)

(72) Inventors: Yexiang Jiang, Guangzhou (CN); Songtao Shi, Guangzhou (CN); Xiaoxing Kou, Guangzhou (CN); Xueli Mao, Guangzhou (CN); Xin Ding, Shenzhen (CN)

(21) Appl. No.: 19/130,881

(22) PCT Filed: Nov. 17, 2023
(86) PCT No.: PCT/CN2023/132236

§ 371 Date: May 16, 2025

(30) Foreign Application Priority Data
Nov. 18, 2022 (CN) 202211447921.3

(51) Int. Cl. A61K 35/28; A61K 31/553; A61K 45/06

(52) U.S. Cl. CPC A61K 35/28; A61K 31/553; A61K 45/06

(57) ABSTRACT

Use of induced vesicles in preparation of a medicament for external procoagulation or external hemostasis. The induced vesicles are obtained by adding an apoptosis inducer to contact stem cells or somatic cells. Wherein, the expression of a tissue factor is the key factor for the induced vesicles to achieve external procoagulation or external hemostasis.

### FIELD

The present disclosure belongs to the field of biomedicines, and relates to the use of vesicles in preparation of a medicament for external procoagulation or external hemostasis.

### BACKGROUND

More than 30% of deaths worldwide are caused by uncontrolled bleeding due to surgery, traffic accidents or war trauma. Therefore, rapid and effective hemostasis is crucial to reduce mortality and morbidity in trauma emergency cases. Conventional hemostasis materials, including hemostasis gauze, hemostasis bandages, gelatin sponges, hemostasis powders, and the like, do not have ideal hemostasis effects. Therefore, the development of a fast, efficient, and safe hemostasis agent is the main goal of current hemostasis materials.

Extracellular vesicles (EVs) are nanoscale particles, which are secreted by cells, contain proteins, nucleic acids, and various cytokines, and have a membrane structure. EVs are generally classified into three categories: exosomes, microvesicles, and apoptotic vesicles. At present, research in this field mainly focuses on exosomes. Exosomes are EVs with a diameter of about 30 to 150 nm, which contain components such as RNA, lipids, and proteins. Studies have shown that exosomes play an important role in maintaining homeostasis and promoting tissue repair. Currently, mesenchymal stem cells (MSCs) are considered to be cells with the strongest ability to produce exosomes. Many studies have revealed that exosomes derived from MSCs have broad application prospects and play an important role in the repair and regeneration of various tissues after damage. However, exosome-based EV therapy still faces many challenges. For example, the extraction and purification process of exosomes is complicated and time-consuming, has high requirements for devices and reagents, and has low physiological exosome yield. These defects limit the clinical transformation and application of exosome therapy.

### SUMMARY

In some embodiments, the present disclosure provides the use of induced vesicles in preparation of a medicament for external procoagulation or external hemostasis.

In some embodiments, the present disclosure provides a method for external procoagulation or external hemostasis, comprising: administering induced vesicles to a patient or a subject.

In some embodiments, the present disclosure provides induced vesicles for external procoagulation or external hemostasis.

In some embodiments, the induced vesicles are tissue factor-positive induced vesicles.

The inventors have found through research that unlike the treatment of hemophilia with induced vesicles, the expression of tissue factor is a key factor in the treatment of external bleeding, and vesicles lacking tissue factor expression will not be able to effectively achieve external hemostasis.

In some embodiments, the present disclosure further provides a method for screening a medicament for external procoagulation or external hemostasis, taking induced vesicles as a candidate medicament to be screened, detecting tissue factor expression in the induced vesicles, and screening tissue factor-positive induced vesicles.

In some embodiments, the present disclosure provides a method for detecting, controlling, or evaluating quality of a medicament for external procoagulation or external hemostasis, taking induced vesicles as a to-be-detected object, a tissue factor expression parameter is taken as an evaluation index, and determining that a medicament is qualified when the tissue factor expression parameter reaches a preset level.

In some embodiments, the tissue factor expression parameter is a tissue factor expression quantity or a tissue factor expression positive rate.

In some embodiments, the tissue factor expression positive rate is a ratio of tissue factor-positive induced vesicles to total induced vesicles.

In some embodiments, the present disclosure provides the use of an induced vesicles product with an improved tissue factor expression positive rate in preparation of a medicament for external procoagulation or external hemostasis.

In some embodiments, the present disclosure provides a method for external procoagulation or external hemostasis, comprising: administering an induced vesicles product with an improved tissue factor expression positive rate to a patient or subject.

In some embodiments, the present disclosure provides induced vesicles with an improved tissue factor expression positive rate for external procoagulation or external hemostasis.

In some embodiments, the induced vesicles product with an improved tissue factor expression positive rate is that a ratio of tissue factor-positive induced vesicles to total induced vesicles is greater than or equal to a preset level.

In some embodiments, the preset level is that: tissue factor expression positive rate ≥30%.

In some embodiments, the preset level is that: tissue factor expression positive rate ≥31%.

In some embodiments, the preset level is that: tissue factor expression positive rate ≥32%.

In some embodiments, the preset level is that: tissue factor expression positive rate ≥35%.

In some embodiments, the preset level is that: tissue factor expression positive rate ≥40%.

In some embodiments, the preset level is that: tissue factor expression positive rate ≥45%.

In some embodiments, the preset level is that: tissue factor expression positive rate ≥50%.

In some embodiments, the preset level is that: tissue factor expression positive rate ≥55%.

In some embodiments, the preset level is that: tissue factor expression positive rate ≥60%.

In some embodiments, the present disclosure provides a composition containing induced vesicles and a tissue factor.

In some embodiments, the composition further contains a procoagulant or a hemostatic.

In some embodiments, the procoagulant or the hemostatic is selected from one or more of prothrombin, thrombin, fibrinogen, fibrin, cohesin, a coagulation factor, collagen, gelatin, vasopressin, a plasminogen activator inhibitor, a platelet activator, and a synthetic peptide with hemostasis activity.

In some embodiments, the present disclosure provides the use of the composition in preparation of a medicament for external procoagulation or external hemostasis.

In some embodiments, the present disclosure provides a method for external procoagulation or external hemostasis, comprising: administering the composition to a patient or subject.

In some embodiments, the present disclosure provides a composition for use in external procoagulation or external hemostasis.

In some embodiments, the present disclosure provides a method for preparing induced vesicles, wherein the method, by adding an apoptosis inducer to contact stem cells or somatic cells, the stem cells or somatic cells producing the induced vesicles, and duration of the apoptosis inducer to contact is about 3-16 h.

In some embodiments, the duration of the apoptosis inducer to contact is about 3-15 h.

In some embodiments, the duration of the apoptosis inducer to contact is about 4-14 h.

In some embodiments, the duration of the apoptosis inducer to contact is about 6-14 h.

In some embodiments, the duration of the apoptosis inducer to contact is about 8-14 h.

In some embodiments, the duration of the apoptosis inducer to contact is about 10-14 h.

In some embodiments, the duration of the apoptosis inducer to contact is about 11-13 h.

In some embodiments, the duration of the apoptosis inducer to contact is about 12 h.

In some embodiments, the method comprises the following steps:
(1) culturing mesenchymal stem cells in a medium;
(2) collecting a supernatant of the medium in (1); and
(3) separating vesicles from the supernatant of the medium in step (2).

In some embodiments, the step of culturing mesenchymal stem cells in step (1) comprises:
(4) separating the mesenchymal stem cells from a tissue; and
(5) adding the medium to culture the mesenchymal stem cells, the mesenchymal stem cells being in contact with the apoptosis inducer in the medium.

In some embodiments, the apoptosis inducer comprises one or a combination of more of staurosporine (STS), ultraviolet irradiation, a starvation method, and a heat stress method.

In some embodiments, the apoptosis inducer is staurosporine.

In some embodiments, a concentration of staurosporine is about 200 nM-1000 nM.

In some embodiments, the concentration of staurosporine is about 250 nM-750 nM.

In some embodiments, the concentration of staurosporine is about 400 nM-600 nM.

In some embodiments, the concentration of staurosporine is about 450 nM-550 nM.

In some embodiments, the concentration of staurosporine is about 500 nM.

In some embodiments, the present disclosure provides the aforementioned method for use in obtaining tissue factor-positive induced vesicles.

In some embodiments, the present disclosure provides the aforementioned method for use in obtaining an induced vesicles product with an improved tissue factor expression positive rate.

In some embodiments, the present disclosure provides induced vesicles prepared by the aforementioned method.

In some embodiments, the present disclosure provides the use of induced vesicles prepared by the aforementioned method in preparation of a medicament for external procoagulation or external hemostasis.

In some embodiments, the present disclosure provides a method for external procoagulation or external hemostasis, comprising: administering induced vesicles prepared by the aforementioned method to a patient or subject.

In some embodiments, the present disclosure provides induced vesicles for external procoagulation or external hemostasis that are prepared by the aforementioned method.

In some embodiments, the induced vesicles are vesicles produced by stem cells in a normal survival state when an external factor is applied to induce apoptosis of the stem cells.

In some embodiments, the stem cells are mesenchymal stem cells.

In some embodiments, the mesenchymal stem cells are derived from bone marrow, dental pulp, urine, oral cavity, adipose, placenta, umbilical cord, periosteum, tendon or peripheral blood.

In some embodiments, the mesenchymal stem cells are umbilical cord mesenchymal stem cells (UC-MSCs).

In some embodiments, the mesenchymal stem cells are derived from a mammal.

In some embodiments, the mammal is selected from a primate or a mouse.

In some embodiments, the primate is a human.

In some embodiments, the medicament is selected from a freeze-dried powder injection, an injection, a nebulized inhalant, an oral preparation, or an external preparation.

In some embodiments, the medicament is an external preparation.

In some embodiments, the external preparation is selected from a tincture, a cataplasm, paste, powder, an ointment, a plaster, a cream, a lotion, liniment, a spray, an aerosol, a film, a patch, gel, liquid, a sponge, or gauze.

In some embodiments, the gel comprises aqueous gel and oleaginous gel.

In some embodiments, the external preparation is a sponge.

In some embodiments, the medicament further comprises a pharmaceutically acceptable carrier.

In some embodiments, the pharmaceutically acceptable carrier comprises one or more of a diluent, an excipient, a filler, a binder, a disintegrant, a surfactant, and a lubricant.

### BRIEF DESCRIPTION OF THE DRAWINGS

FIG. 1 Flow cytometry results of cell surfaces of umbilical cord mesenchymal stromal cells (UC-MSCs).

FIG. 2 Procoagulant effects of induced extracellular vesicles (IEVs) respectively subjected to phosphatidylserine (PS) blocking, tissue factor (TF) blocking, and TF knockdown. A. comparison of procoagulant effects of IEVs after antibody blocking of PS and TF; B. comparison of procoagulant effects of IEVs after TF knockdown by siRNA. (****p<0.0001).

FIG. 3 Scanning electron microscopy (SEM) of IEVs promoting stability of a blood clot. A. SEM images of outer surfaces of blood clots; B. proteomics comparison of IEVs and exosomes (exo); C. efficiency of siRNA knockdown verified by the western blot; D. SEM images of inner and outer surfaces of blood clots.

FIG. 4 Whole blood clotting time of different counts of IEVs. (*p<0.05, **p<0.01, and ***p<0.001).

FIG. 5 Flow cytometry detecting TF and PS expression of IEVs obtained by inducing MSCs with different concentrations of STS for different induction time.

FIG. 6 Comparison of whole blood clotting time of IEVs. A. comparison of whole blood clotting time of fresh IEVs and freeze-dried IEVs; B. comparison of whole blood clotting time of IEVs stored at −80 °C, 4 °C, and 25 °C, respectively, for 28 d. (****p<0.0001, ns: not significant).

FIG. 7 Effect of IEVs/gelatin sponge composite on a rat femoral artery bleeding model. A. photo of IEVs/gelatin sponge composite; B. Sprague Dawley (SD) rat femoral artery bleeding model; C. 10-s free blood loss of superficial femoral artery and vein in each group; and D. 10-min compression-induced blood loss of each group. (*p<0.05, **p<0.01, ns: not significant).

FIG. 8 Effect of IEVs/gelatin sponge composite on a New Zealand White Rabbit femoral artery bleeding model. A. New Zealand White Rabbit femoral artery bleeding model; B. femoral artery of New Zealand White Rabbit; C. 10-s free blood loss of the femoral artery in each group; D. 10-min compression-induced blood loss of each group. (*p<0.05, ns: not significant).

FIG. 9 Comparison of wound healing outcomes in mice.

FIG. 10 Comparison of wound healing rates in mice.

FIG. 11 Whole blood clotting time of IEVs derived from different stem cells. (*p<0.05, ****p<0.0001, ns: not significant).

FIG. 12 Promoting effect of IEVs/gelatin sponge composite on oral mucosal wound healing in a rat and a hemostasis effect on bleeding after tooth extraction in a rat. A. image of oral wounds of rats in each group at different time points; B. oral mucosal wound area of rats in each group at different time points; C. hemostasis time and blood loss of rats in each group after tooth extraction. (*p<0.05, **p<0.01, ns: not significant).

FIG. 13 Hemostasis effect of IEVs/gelatin sponge composite on bleeding after mandibular wisdom tooth extraction in a human. A. surgical procedure of mandibular third molar extraction in a human; B. hemostasis time after mandibular wisdom tooth extraction in a human. (*p<0.05).

FIG. 14 Preparation and characterization of IEVs-hydrogel sponge. A. preparation process of an IEVs-HS; B. digital photo of IEVs-hydrogel sponge C. swelling property of IEVs-hydrogel sponge; D. adhesion strength of IEVs-hydrogel sponge to the heart, liver, spleen, lung, kidney or skin tissue of the mouse; E. blood clotting index (BCI) test; F. in vitro IEVs release profile of IEVs-hydrogel sponge; G. SEM image.

FIG. 15 Hemostasis effect of IEVs-hydrogel sponge on liver bleeding in a rat. A. surgical procedure of the rat liver bleeding model; B. hemostasis time of rats in each group; C. blood loss of rats in each group; D. hematoxylin and eosin (HE) staining of the livers of rats in each hemostasis material group; E. Immunofluorescence image of CD3 cells in the liver of rats in each hemostasis material group.

FIG. 16 Effect of IEVs-hydrogel sponge on rat femoral artery bleeding model. A. surgical procedure of the rat femoral artery bleeding model; B. hemostasis time of the rat femoral artery bleeding model in each group; C. blood loss of the rat femoral artery bleeding model in each group. (*p<0.05, **p<0.01, ***p<0.001, ns: not significant).

FIG. 17 Effect of IEVs-hydrogel sponge on New Zealand White Rabbit femoral artery bleeding model. A. surgical procedure of the New Zealand White Rabbit femoral artery bleeding model; B. hemostasis time of New Zealand White Rabbits in each group; C. blood loss of New Zealand White Rabbits in each group. (*p<0.05, **p<0.01, ***p<0.001, ns: not significant).

### DETAILED DESCRIPTION

The technical solutions of the present disclosure will be further described with reference to specific examples, but the specific examples are not intended to limit the scope of protection of the present disclosure. Some non-essential modifications and adjustments made by others based on the concepts of the present disclosure still fall within the scope of protection scope of the present disclosure.

In the examples of the present disclosure, IEVs stands for induced vesicles. induced vesicles refer to a type of subcellular product secreted by precursor cells (such as stem cells) in a normal survival state when the precursor cells are intervened or induced to undergo apoptosis. Typically, the subcellular products have a membrane structure and express an apoptotic marker, and some contain genetic material DNA. The inventors have found that the induced vesicles s are substances distinguished from cells and conventional extracellular vesicles (such as exosomes). In some embodiments, the cells in the normal survival state are, for example, non-apoptotic cells, non-senescent cells, non-aging cells with arrested proliferation, non-frozen and thawed cells, non-malignant and abnormally proliferating cells, or non-damaged cells. In some embodiments, the cells in the normal survival state are selected from cells when a cell contact fusion rate reaches 80% to 100% during cell culture. In some embodiments, the cells in the normal survival state are selected from cells in the logarithmic phase. In some embodiments, the cells in the normal survival state are selected from primary culture and subculture cells derived from a human or mouse tissue.

In some embodiments, the cells in the normal survival state are selected from an established cell line or cell strain. In some embodiments, the precursor cells are selected from early cells. In some embodiments, the EVs may also derived from P3 generation to P8 generation umbilical cord mesenchymal stromal cells (UC-MSCs).

In the present disclosure, STS stands for staurosporine.

In the present disclosure, TF stands for tissue factor.

As used herein, the term tissue factor expression parameter refers to a tissue factor expression quantity or a tissue factor expression positive rate.

As used herein, the term tissue factor expression positive rate refers to a ratio of tissue factor-positive induced vesicles to total induced vesicles.

Include, comprise, or contain is intended to indicate that combinations (such as a medium) and methods include the listed elements but not exclude other elements. When used to define compositions and methods, essentially consisting of is meant to exclude other elements of any significance to the combination for the stated purpose. Therefore, a composition essentially consisting of the elements defined herein does not exclude other materials or steps that do not materially affect the basic and novel characteristics of the present disclosure claimed. Consisting of means excluding trace elements of other ingredients and substantial process steps. Embodiments defined by each of these transition terms fall within the scope of the present disclosure.

### Example 1 Separation and Culture of Umbilical Cord-Mesenchymal Stem Cells (UC-MSCs)

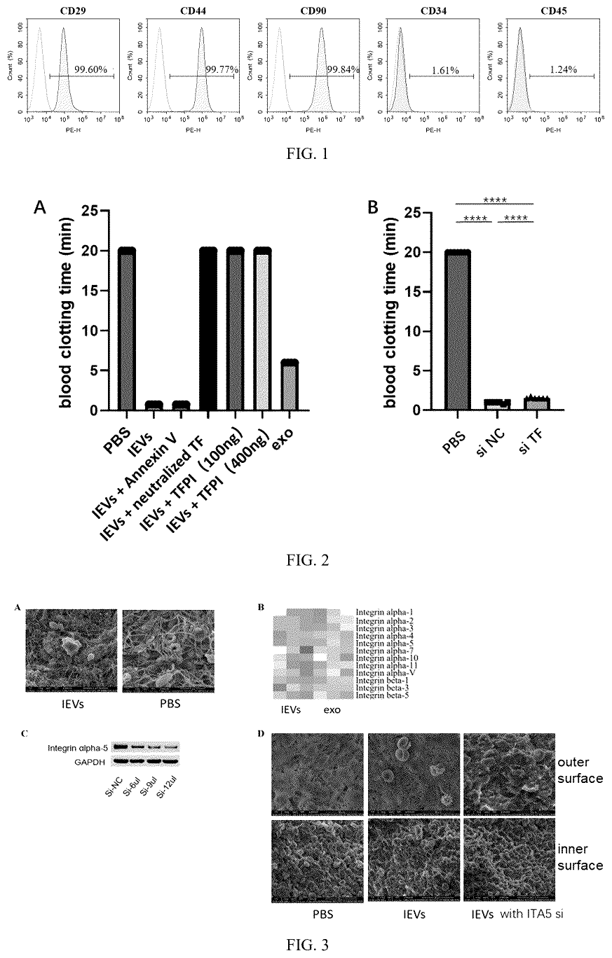

First, the umbilical cord (obtained from Department of Obstetrics and Gynecology, Guangdong Provincial People's Hospital) was washed with phosphate-buffered saline (PBS) containing penicillin and streptomycin to remove blood. The washed umbilical cord was cut into segments having a length of 3-4 cm in a culture dish, and the umbilical cord blood vessels were removed. Then, the umbilical cord was cut into small pieces, and digested in PBS containing 2 mg/mL collagenase type I and 4 mg/mL dispase II at 37 °C for 1 h. The digested tissue was plated in a 100 mm culture dish containing complete medium. Then, cells were incubated at 37 °C and 5% carbon dioxide for 72 h, and cultures were washed twice with PBS to remove nonadherent cells. The complete medium was replaced with a new complete medium, after 7-10 days, dense primary UC-MSCs colonies were visible. Characteristic markers of the UC-MSCs were detected by using a flow cytometer. Results demonstrate that CD29, CD44, and CD90 (≥95%) are expressed and CD34 and CD45 (≤5%) are not expressed in the obtained UC-MSCs (FIG. 1). The UC-MSCs were incubated and digested with trypsin at 37 °C, and subcultured and amplified, the complete medium was replaced every three days, and the cells were subcultured after confluence. A composition of the complete medium is shown in Table 1.

TABLE 1
Composition of complete medium

| | Final | | | |
|---|---|---|---|---|
| Reagent | concentration | | Manufacturer | Catalog No. |
| FBS | 15% | | ExcellBio | FSP500 |
| Penicillin/streptomycin | 100 | ug/mL | GIBCO | 15140122 |
| solution | | | | |
| Glutamine | 2 | mM | Invitrogen | 35050079 |
| Ascorbic acid | 0.1 | mM | Wako | 013-12061 |

| | | | |
|---|---|---|---|
| α-MEM | 82% | Biosharp | BL306A |

### Example 2 Acquisition of IEVs Derived from UC-MSCs

The UC-MSCs may be P3 to P8 generation cells, but are not limited thereto. In this example, P7 generation UC-MSCs are selected.

The P7 generation UC-MSCs with a confluence rate of 80% to 90% culture in Example 1 were selected, washed with PBS, added to an alpha medium containing 500 nM staurosporine (STS), and incubated at 37 °C for 12 h, a cell supernatant was collected and centrifuged at 4 °C and 800 g for 10 min, a supernatant was collected and centrifuged at 4 °C and 2000 g for 10 min, a supernatant was collected and centrifuged at 4 °C and 16000 g for 30 min, and a supernatant was discarded to obtain a precipitate, namely, IEVs. The IEVs were resuspended in 500 μL of PBS and centrifuged at 4 °C and 16000 g for 30 min, a supernatant was discarded to obtain washed IEVs.

### Example 3 Role of TF and Phosphatidylserine (PS) in Procoagulant Mechanism of IEVs

IEVs treated with antibody blocking and small interfering RNA (siRNA) knockdown techniques were used for plasma clotting tests to explore the procoagulant mechanism of IEVs.

(1) Plasma clotting test: 12.5 μL of 1×10⁸ IEVs was added to a flow tube (in addition, a control group was set: 12.5 μL of PBS was placed in a flow tube), pre-heated at 37 °C for 10 min, and 40 μL of mouse plasma was added, and the mixture was uniformly mixed. 7 μL of 0.2 M CaCl₂) solution was added, and timing was started immediately. The flow tube was shaken up and down once every 15 s, and time when the plasma clotted and could not be shaken was recorded as blood clotting time.

(2) Antibody blocking of TF and PS: the IEVs obtained in Example 2 were resuspended in PBS, and a count of IEVs was measured by using a nanoparticle tracking analyzer (Particle Metrix, Germany). 2×10⁸ IEVs were picked and respectively incubated with 1 μg/mL TF blocking antibody (neutralized TF, FAB3178P, R&D Systems, U.S.A.), 1 μg/mL tissue factor pathway inhibitor (TFPI) (PU299161, Abmart, China), and 1 μg/mL Annexin V (10039-01, Sino Biological Inc, China) at 4 °C for 30 min. The IEVs subjected to antibody blocking were directly used for plasma clotting tests. Results demonstrate that blocking of PS does not affect the procoagulant function of the IEVs, but procoagulant effects of the IEVs treated with the TF neutralizing antibody and the TFPI are significantly reduced (FIG. 2A).

(3) TF knockdown: TF expression in IEVs derived from UC-MSCs was eliminated by an siRNA technology. An siRNA negative control (si-NC) and an siRNA-TF (si-TF) were respectively transfected into the UC-MSCs by using a Lipofectamine™ RNAiMAX transfection reagent (Invitrogen, U.S.A.) in accordance with instructions of the manufacturer. Transfection efficiency was measured by the western blot after 48 h of transfection. IEVs were extracted from UC-MSCs transfected with siRNA for 72 h for plasma clotting tests. IEVs extraction steps and conditions were the same as those in Example 2, and the plasma clotting tests were conducted according to step (1). Results demonstrate that compared to the control group, a procoagulant effect of the TF knockdown IEVs is significantly reduced, and a difference is statistically significant (FIG. 2B).

The foregoing test results indicate that the IEVs mainly promote rapid procoagulant through the TF. This is different from the performance and mechanism of IEVs in treating hemophilia disclosed in CN114306238A. CN114306238A disclosed that the mechanism of IEVs in treating hemophilia in mice is unassociated with TF and PS. This indicates that the procoagulant mechanism of the present disclosure is different from the hemophilia treatment mechanism.

### Example 4 Scanning Electron Microscopy Detection of IEVs Promoting Blood Clot Stability

The IEVs obtained in Example 2 were selected. 50 μL of 1×10¹⁰ IEVs were placed at the bottom of a 48-well plate (in addition, a PBS control group was set: 50 μL of PBS was placed at the bottom of a 48-well plate), and pre-heated at 37 °C for 10 min. 0.5 mL of citrate anticoagulated rat whole blood was added to each well, and the mixture was uniformly mixed. 50 μL of 0.2 mol/L CaCl₂) was added, and incubation was performed at 37 °C for 1 h. The sample was fixed with 2 mL of 2.5% (v/v) glutaraldehyde at 4 °C for 4 to 6 h. The sample was dehydrated with 50%, 70%, 85%, and 100% ethanol in sequence for 10 min, dried, and surface-coated with gold, and finally, observed using a scanning electron microscope.

SEM images show that the surface of a blood clot in the PBS group has loosely interwoven fibers and large pores, while the surface of a blood clot in the IEVs group have fine and dense fibers and small pores (FIG. 3A). Quantitative proteomics analysis of the IEVs and exosomes is performed by the data-independent acquisition (DIA) quantitative proteomics technology. Results demonstrate that compared to exosomes derived from the same type of MSCs, the IEVs are rich in integrin α5 (FIG. 3B), which is one of the important substances binding to fibrin. Therefore, integrin α5 in P5 generation UC-MSCs was knocked down by the siRNA technology to obtain IEVs with low integrin α5 expression (FIG. 3C), so as to clarify whether the IEVs stabilized fibrin via integrin α5. SEM images show that the inner and outer surfaces of the blood clots in the IEVs group with low integrin α5 expression have more loosely arranged fibers and larger pore sizes compared to those in the IEVs group with normal integrin α5 expression (FIG. 3D). Results demonstrate that IEVs make fibrin in blood clots denser via integrin α5, thereby promoting stability of the blood clots.

In the quantitative proteomics analysis, the exosomes and the IEVs were extracted from a same cell strain.

A separation and extraction method of exosomes: P7 generation UC-MSCs with a convergence rate of 80% to 90% cultured in Example 1 were selected, washed with PBS, added to a serum-free medium (α-MEM medium), and incubated at 37 °C for 48 h, a cell supernatant was collected for separation and extraction of exosomes. Extraction process included: the supernatant was centrifuged at 800 g for 10 min, a supernatant was collected and centrifuged at 2000 g for 10 min, a supernatant was collected and centrifuged at 16000 g for 30 min, a supernatant was collected and centrifuged at 120000 g for 90 min, a supernatant was removed, a precipitate was resuspended in sterile PBS and centrifuged at 120000 g for 90 min, a supernatant was removed, and exosomes at the bottom were collected and resuspended in sterile PBS.

### Example 5 Comparison of Blood Clotting Time by Different Counts of IEVs Derived from UC-MSCs

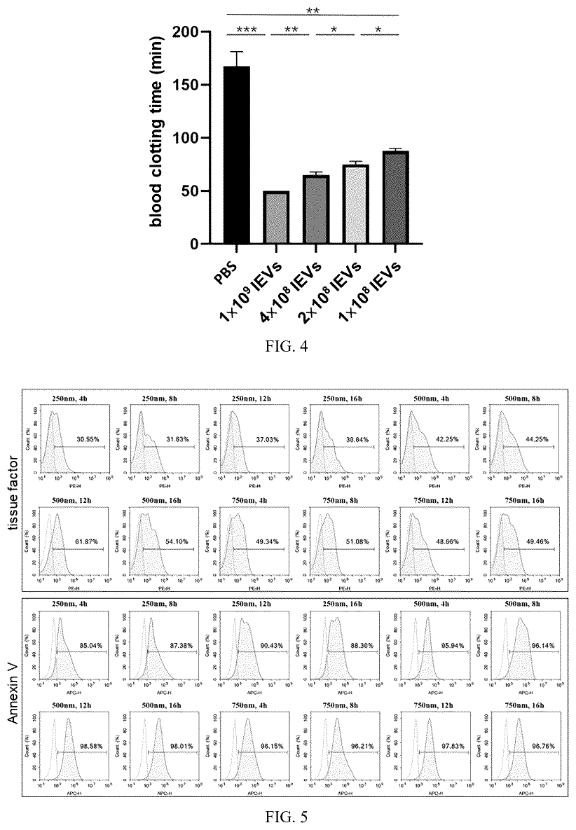

The IEVs obtained in Example 2 were selected. 1×10⁹ IEVs, 4×10⁸ IEVs, 2×10⁸ IEVs, and 1×10⁸ IEVs were used for the whole blood clotting test. The whole blood clotting test was conducted as follows: 500 μL of IEVs resuspended in PBS was placed in a flow tube (a blank control group: 500 μL of PBS was placed in a flow tube), pre-heated at 37 °C for 10 min, and 1 mL of sodium citrate anticoagulated rat whole blood was added, after the mixture was uniformly mixed, 100 μL of 0.1 M CaCl₂) solution was added, timing was started immediately, the flow tube was shaken up and down once every 10 s, and time when the blood clotted and could not be shaken was recorded as blood clotting time. Test results demonstrate that with the reduction of the count of IEVs, the blood clotting time is prolonged, and a difference is statistically significant, which indicates that the procoagulant performance of the IEVs is dose-dependent (FIG. 4).

### Example 6 Acquisition of IEVs Derived from UC-MSCs Under Different Conditions

P7 generation UC-MSCs with a convergence rate of 80% to 90% cultured in Example 1 were selected, and washed with PBS, alpha media respectively containing 250 nM, 500 nM, and 750 nM staurosporine (STS) were added, the cells were incubated at 37 °C, a cell supernatant was collected after 4 h, 8 h, 12 h, and 16 h, respectively, the supernatants collected at different time points were respectively subjected to the following operations: the supernatant was centrifuged at 4 °C and 800 g for 10 min, a supernatant was collected and centrifuged at 4 °C and 2000 g for 10 min, a supernatant was collected and centrifuged at 4 °C and 16000 g for 30 min, and a supernatant was discarded to obtain a precipitate, namely, IEVs. The IEVs were resuspended in 500 μL of PBS and centrifuged at 4 °C and 16000 g for 30 min, a supernatant was discarded to obtain washed IEVs.

### Example 7 TF and PS Expression in IEVs Derived from UC-MSCs

The IEVs obtained under different concentrations of STS and different induction time in Example 6 were selected. The IEVs were resuspended in PBS, and a count of IEVs was measured by using a nanoparticle tracking analyzer (Particle Metrix, Germany). 1×10¹⁰ IEVs were resuspended in 98 μL of FACS buffer, 2 μL of phycoerythrin (PE)-conjugated anti-human TF (BioLegend, U.S.A.), and allophycocyanin (APC)-conjugated Annexin V (BioLegend, U.S.A.) were respectively added, and incubation was performed at 4 °C for 30 min. A percentage of positive staining was analyzed by using a NovoCyte flow cytometer (ACEA Biosciences, U.S.A.) and NovoExpress™ software (NovoCyte, U.S.A.). Results demonstrate that TF expression and PS expression in the IEVs obtained after induction with 500 nM STS for 12 h are the highest (FIG. 5).

### Example 8 Preparation of Frozen-Dried IEVs Derived from UC-MSCs

The IEVs obtained in Example 2 were selected and resuspended in 100 μL of PBS, 400 μL of frozen-drying solution was added, and the mixture was uniformly mixed. A composition of the frozen-drying solution was as follows: 4 mL of 0.65×PBS contained 100 mM trehalose and 5% polyvinylpyrrolidone 40 (PVP40). The mixture of the IEVs and the freeze-drying solution was placed in a refrigerator at −80 °C overnight, the frozen sample was taken out of the refrigerator at −80 °C the next day, and the EP tube containing the mixture of the IEVs and the freeze-drying solution was quickly opened and covered with plastic wrap, and then placed in a −80 °C freeze dryer (FD-A18N-80, Shanghai Shengwei Electronic Technology), and freeze-dried for 30 h to obtain freeze-dried IEVs.

### Example 9 Comparison of Blood Clotting Time of Fresh IEVs, Freeze-Dried IEVs, and Freeze-Dried IEVs Placed at Room Temperature for 28 d

Whole blood clotting test: a total of five groups were set, which were fresh IEVs group (1×10⁹ IEVs, and 500 μL of PBS), freeze-dried IEVs group (1×10⁹ IEVs, and 500 μL of PBS), fresh PBS group (500 μL of PBS), freeze-dried PBS group (freeze-dried PBS, 500 μL of PBS), and freeze-drying solution group (500 μL of freeze-drying solution), and the whole blood clotting test was conducted according to the operations in Example 5.

The fresh IEVs were the IEVs obtained in Example 2, the freeze-dried IEVs were the IEVs prepared in Example 8, the freeze-dried PBS was prepared by the steps in Example 8, and a difference was that no IEVs were added.

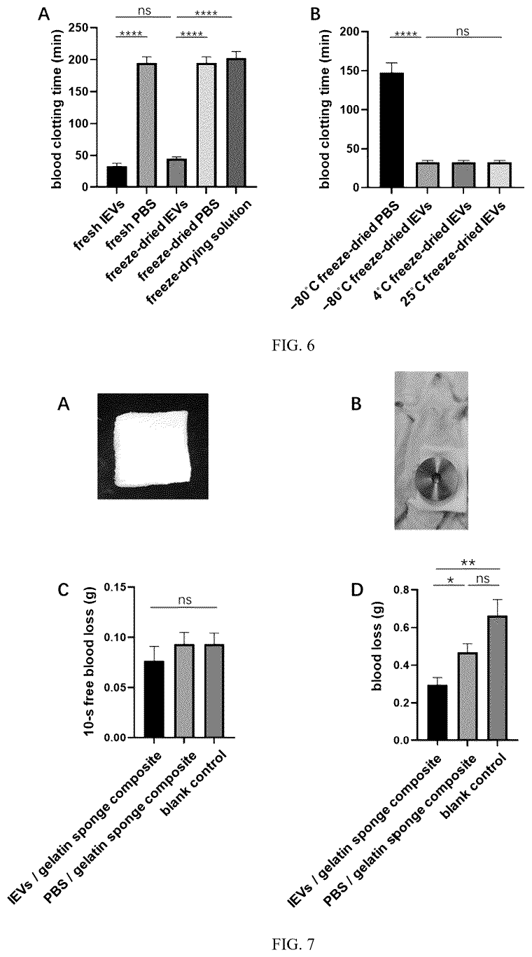

Results demonstrate that the blood clotting time of the freeze-dried IEVs group is significantly reduced compared to the blood clotting time of the PBS group, and a difference in procoagulant effects between the freeze-dried IEVs and the fresh IEVs is not statistically significant (FIG. 6A). The freeze-dried IEVs were stored at −80 °C, 4 °C, and 25 °C, respectively, for 28 d. Results demonstrate that a procoagulant effect of the freeze-dried IEVs placed at room temperature for 28 d is still high, and a difference in the procoagulant effects between the freeze-dried IEVs placed at room temperature and the freeze-dried IEVs stored at −80 °C or 4 °C is not statistically significant (FIG. 6B). The foregoing results indicate that the rapid procoagulant effect of the IEVs prepared by the freeze-drying process is maintained, which may solve the problems related to transportation, carrying, and storage in future use.

### Example 10 Hemostasis Effect of Freeze-Dried IEVs/Gelatin Sponge Composite on Femoral Artery Bleeding in Rat

### 1. Preparation of Freeze-Dried IEVs/Gelatin Sponge Composite

The IEVs obtained in Example 2 were selected and resuspended in 100 μL in PBS, 400 μL of freeze-drying solution was added (with the same composition as that in Example 8), the mixture was uniformly mixed, and 500 μL of mixture was added to a gelatin sponge with a size of 20 mm×20 mm (Xiangen Medical, China). After the mixture was completely absorbed by the gelatin sponge, the gelatin sponge was placed in a refrigerator at −80 °C overnight, and freeze-dried in a freeze-dryer at −80 °C for 30 h the next day, to obtain a freeze-dried IEVs/gelatin sponge composite. Preparation of PBS/gelatin sponge composite: no IEVs were added, and other steps were the same as that of the preparation method of the freeze-dried IEVs/gelatin sponge composite.

### 2. Construction and Grouping of Rat Bleeding Models

250±20 g Sprague Dawley (SD) rats were randomly divided into the following groups according to the weight. The groups are: IEVs/gelatin sponge composite group, PBS/gelatin sponge composite group, and blank control group, 5 rats per group. Experimental procedure: the rat was shaved, the superficial femoral artery and vein were isolated, and a surgical area was wiped clean with gauze to prevent exudate from interfering with experimental results. The superficial femoral artery and vein were clamped with an artery clamp, and cut with scissors, the artery clamp was loosened, and the blood vessel was allowed to bleed freely for 10 s and then pressed with a piece of pre-weighed gauze and a 200 g weight for 10 s. Then, the IEVs/gelatin sponge composite and the PBS/gelatin sponge composite that had a same weight were respectively placed on the bleeding point. For the blank control group, no gelatin sponge was placed, and the bleeding point was covered with 3 pieces of pre-weighed gauze and pressed with a 200 g weight for 10 m. The blood loss was calculated by using the following formula: blood loss=weight of gauze after hemostasis−original weight of gauze. A difference in the 10-s free blood losses between the groups is not statistically significant, indicating that the degree of bleeding modeling is consistent (FIG. 7C). The blood loss of the IEVs/gelatin sponge composite group is significantly less than that of the PBS/gelatin sponge composite group or the blank control group, and a difference is statistically significant, indicating efficacy of the freeze-dried IEVs/gelatin sponge composite.

### Example 11 Hemostasis Effect of Freeze-Dried IEVs/Gelatin Sponge Composite on Femoral Artery Bleeding in New Zealand White Rabbit

Preparation of the freeze-dried IEVs/gelatin sponge composite and the PBS/gelatin sponge composite was the same as that in Example 10.

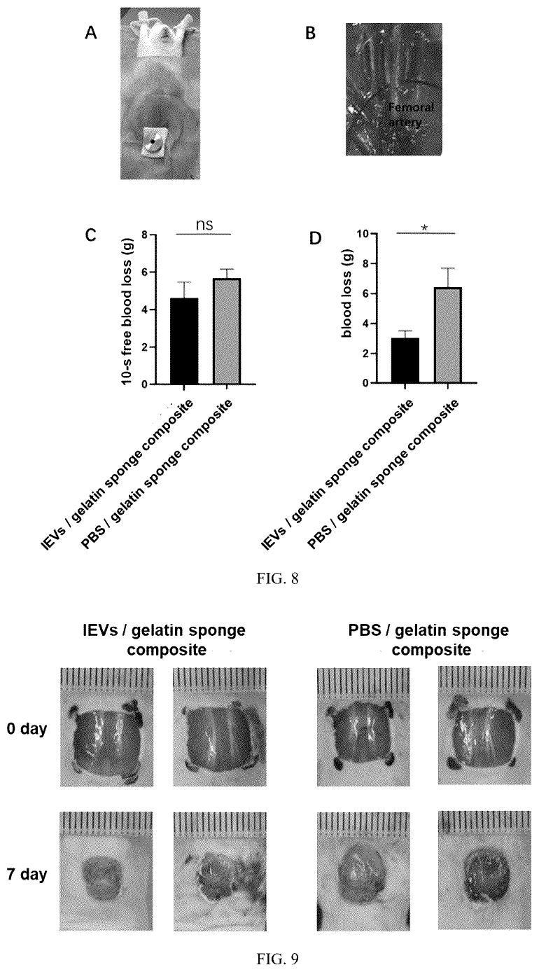

2000±200 g New Zealand White Rabbits were randomly divided into two groups, namely, IEVs/gelatin sponge composite group and PBS/gelatin sponge composite group, 5 rabbits per group. Experimental procedure: the rabbit was shaved, the femoral artery was isolated, and a surgical area was wiped clean with gauze to prevent exudate from interfering with experimental results. The femoral artery was clamped with an artery clamp, a hole (with a diameter of 0.5 mm) was poked in the femoral artery with a tip of a 1 mL needle, the artery clamp was loosened, and the blood vessel was allowed to bleed freely for 10 s and then pressed with 3 pieces of pre-weighed gauze and a 200 g weight for 10 s. Then, 2 IEVs gelatin sponges or PBS gelatin sponges were placed on the bleeding point, and the bleeding point was covered with 5 pieces of pre-weighed gauze, and pressed with a 200 g weight. Pressing was suspended and a hemostasis effect was checked after 3 min, 5 min, 7 min, 9 min, 11 min, 13 min, and 15 min, respectively. The blood loss was calculated by using the following formula: blood loss=weight of gauze after hemostasis-original weight of gauze. A difference in the 10-s free blood losses between the two groups is not statistically significant, indicating that the degree of bleeding modeling is consistent (FIG. 8C). The blood loss of the IEVs/gelatin sponge composite group is significantly less than that of the PBS/gelatin sponge composite group, and a difference is statistically significant, indicating efficacy of the freeze-dried IEVs/gelatin sponge composite (FIG. 8D).

### Example 12 Promoting Effect of Freeze-Dried IEVs/Gelatin Sponge Composite on Wound Healing in Mouse

Preparation of the freeze-dried IEVs/gelatin sponge composite and the PBS/gelatin sponge composite was the same as that in Example 10.

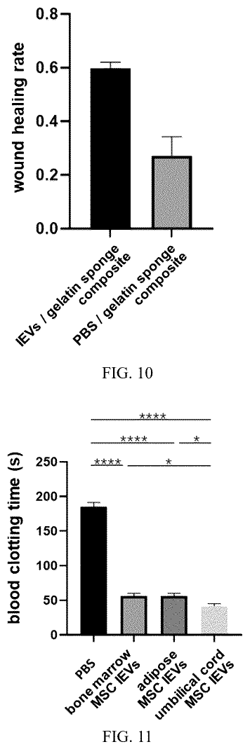

10-week-old C57 mice were randomly grouped according to the weight. A 1 cm×1 cm full-thickness wound was made on the back of each mouse, and a freeze-dried IEVs/gelatin sponge composite or a freeze-dried PBS/gelatin sponge composite was attached to the wound, and then the material was fixed with adhesive tape. On day 7, the freeze-dried IEVs/gelatin sponge composite or the freeze-dried PBS/gelatin sponge composite was removed and the wounds were photographed (FIG. 9), and a wound healing rate of the mice was calculated (FIG. 10). Results demonstrate that the freeze-dried IEVs/gelatin sponge composite can significantly improve the wound healing rate.

### Example 13 Comparison of Blood Clotting Time of IEVs Derived from Different Stem Cells

(1) Collection of IEVs derived from different stem cells: P8-P10 generation human bone marrow mesenchymal stem cells (ScienCell, U.S.A.), human adipose mesenchymal stem cells (Procell, U.S.A.), and human umbilical cord mesenchymal stem cells (UC-MSCs) (Guangdong Provincial People's Hospital, China) were respectively added to an alpha medium containing 500 nM staurosporine (STS), incubated at 37 °C, and induced for 12 h, a cell supernatant was collected and centrifuged at 4 °C and 800 g for 10 min, a supernatant was collected and centrifuged at 4 °C and 2000 g for 10 min, a supernatant was collected and centrifuged at 4 °C and 16000 g for 30 min, a supernatant was discarded, a precipitate was resuspended in 500 μL of PBS and centrifuged at 4 °C and 16000 g for 30 min, and a supernatant was discarded to obtain IEVs derived from different cells.

(2) Whole blood clotting test: 1×10⁹ IEVs resuspended in 500 μL of PBS was placed in a flow tube (a blank control group: 500 μL of PBS was placed in a flow tube) and pre-heated at 37 °C for 10 min, 1 mL of sodium citrate anticoagulated rat whole blood was added, the mixture was uniformly mixed, 100 μL of 0.1 M CaCl₂) solution was added, timing was started immediately, the flow tube was shaken up and down once every 10 s, and time when the blood clotted and could not be shaken was recorded as blood clotting time. Test results demonstrate that the blood clotting time of the IEVs derived from the UC-MSCs is the shortest (FIG. 11).

### Example 14 Promoting Effect of Freeze-Dried IEVs/Gelatin Sponge Composite on Oral Mucosal Wound Healing in Rat

Preparation of the freeze-dried IEVs/gelatin sponge composite and the PBS/gelatin sponge composite was the same as that in Example 10.

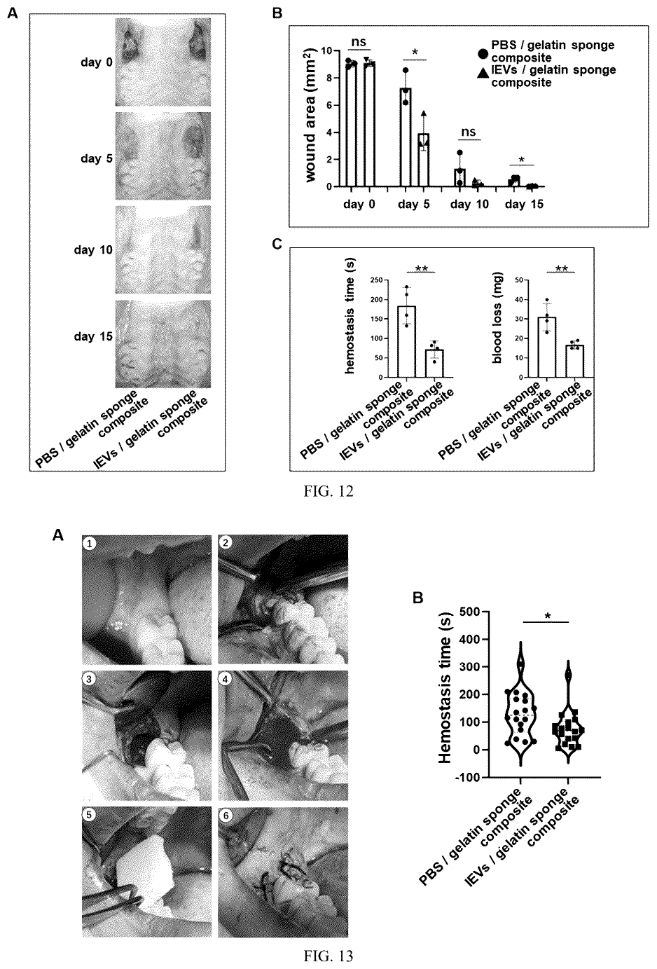

250±20 g SD rats were randomly grouped according to the weight, 12 rats per group. The first molars on both sides of each rat were extracted, a freeze-dried IEVs/gelatin sponge composite or a freeze-dried PBS/gelatin sponge composite was pasted into the extraction socket, and then the gelatin sponge material was fixed with tissue glue. On day 5, day 10, and day 15, three rats in each group were sacrificed, and the oral wounds were photographed (FIG. 12A) to observe the wound areas of the oral mucosa of the rats at different time points. Results demonstrate that the freeze-dried IEVs/gelatin sponge composite significantly promotes oral mucosal wound healing in the rats on day 5 and day 15 (FIG. 12B).

### Example 15 Hemostasis Effect of Freeze-Dried IEVs/Gelatin Sponge Composite on Bleeding after Tooth Extraction in Rat

Preparation of the freeze-dried IEVs/gelatin sponge composite and the PBS/gelatin sponge composite was the same as that in Example 10.

2-month-old SD rats were randomly grouped according to the weight, 4 rats per group. The first molars on both sides of each rat were extracted, the freeze-dried IEVs/gelatin sponge composite or the freeze-dried PBS/gelatin sponge composite was pasted into the extraction socket, and the hemostasis time was observed and recorded. Results demonstrate that the freeze-dried IEVs/gelatin sponge composite can significantly reduce the hemostasis time and the blood loss after tooth extraction in the rats (FIG. 12C).

### Example 16 Hemostasis Effect of Freeze-Dried IEVs/Gelatin Sponge Composite on Bleeding after Mandibular Wisdom Tooth Extraction in Human

Preparation of the freeze-dried IEVs/gelatin sponge composite and the PBS/gelatin sponge composite was the same as that in Example 10.

A total of 19 patients with symmetrical mandibular wisdom teeth on both sides were included in the experiment. After the mandibular third molars on both sides were extracted under local anesthesia, a piece of freeze-dried IEVs/gelatin sponge composite or a freeze-dried PBS/gelatin sponge composite was placed into the alveolar socket for hemostasis and suture, and the patients were followed up (FIG. 13A). In the experimental group, each IEVs/gelatin sponge composite contains 4×10¹⁰ IEVs. The study was a self-controlled experiment.

Patient inclusion criteria were: 1) patients aged 18-45 years (inclusive), regardless of gender; 2) patients who needed extraction of fully impacted mandibular third molars on both sides; and 3) patients who had signed the informed consent form.

Patient exclusion criteria were: 1) patients whose adjacent second molars affected by pulp damage or inadequate pulp treatment, or showing tooth fracture, or with 3 degrees of mobility, and usually with a hopeless prognosis in comprehensive clinical and imaging evaluation; 2) patients whose adjacent second molars with metal crowns or large amalgam restorations that interfere with imaging evaluation; 3) patients with uncontrollable pathological processes in the oral cavity (acute progressive diseases in the oral cavity); 4) patients with malnutrition (serum albumin concentration <2 g/dl); 5) patients diagnosed with other systemic, infectious, and genetic diseases, or patients in the acute inflammatory stage and acute exacerbation of chronic diseases; 6) patients who were pregnant, lactating or planning to become pregnant; 7) patients deemed unsuitable for this study by the researchers.

Results demonstrate that the freeze-dried IEVs/gelatin sponge composite can significantly reduce the hemostasis time after mandibular wisdom tooth extraction in the human (FIG. 13B).

### Example 17 Preparation and Characterization of IEVs Hydrogel Sponge (IEVs-HS)

### 1. Preparation of Phenylboronic Acid-Modified Oxidized Hyaluronic Acid (PBA-HA)

1 g of sodium hyaluronate (Yuanye Bio-technology) was dissolved in 100 mL of deionized water by stirring (200 rpm), and mixed with 3-aminophenylboronic acid (Macklin, China) and 4-(4,6-dimethoxy-1,3,5-triazine-2-yl)-4-methylmorpholinium chloride (Macklin, China) in a molar ratio of 1:1:1. The mixture was stirred at the room temperature for 24 h (200 rpm), and dialyzed with deionized water for 3 d to eliminate unreacted substances. The dialyzed solution was freeze-dried in a freeze-dryer (Labconco, U.S.A.) at −50 °C to obtain a white spongy solid, namely, phenylboronic acid-modified hyaluronic acid. 1 g of phenylboronic acid-modified hyaluronic acid was dissolved in 100 mL of deionized water, and then 10 mL of 0.25 M sodium periodate (Macklin, China) was dropwise added. After 24 h of reaction at room temperature in dark, 1 mL of ethylene glycol (Macklin, China) was added to terminate the reaction, and then the mixture was transferred to a dialysis membrane (MWCO 3,500 Da) to purify the phenylboronic acid-modified oxidized hyaluronic acid. After 3 d of deionization (DI) dialysis, the purified material was freeze-dried in a freeze-dryer at −50 °C and stored at 4 °C.

### 2. Synthesis of IEVs Hydrogel Sponge

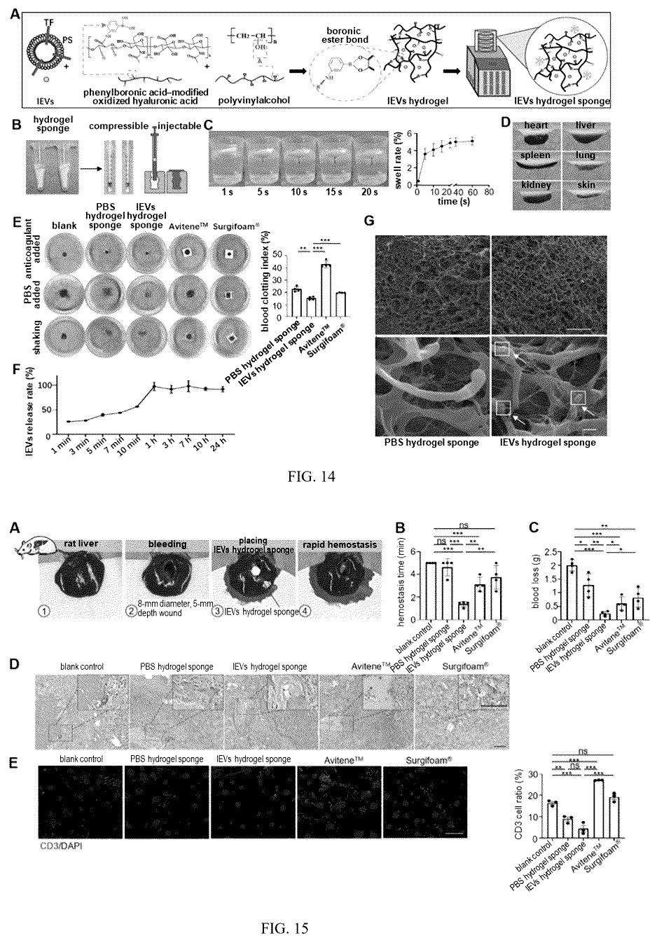

60 mg of phenylboronic acid-modified oxidized hyaluronic acid was dissolved in 500 μL of PBS at room temperature for 1 h. 40 mg of polyvinyl alcohol (Aladdin, China) was dissolved in 500 μL of PBS and placed at 95 °C for 1 h. 50 μL of IEVs (4×10¹⁰ IEVs obtained in Example 2) or 50 μL of PBS was added to a freeze-drying protective agent (200 μL) containing 200 mM trehalose and 10% PVP40, and 125 μL of phenylboronic acid-modified oxidized hyaluronic acid solution and 125 μL of polyvinyl alcohol solution to prepare an IEVs hydrogel or PBS hydrogel. The hydrogel was frozen at −80 °C overnight, and then freeze-dried in a freeze-dryer at −80 °C for 30 h to obtain an IEVs hydrogel sponge or PBS hydrogel sponge (FIG. 14A).

### 3. Water Absorption and Adhesion Tests

60 mg of IEVs hydrogel sponge was compressed and injected into deionized water to prepare hydrated IEVs hydrogel. After 10 min, excess water was removed, and the IEVs hydrogel was placed on a glass slide (Citotest, China). Then, a heart, liver, spleen, lung, kidney or skin tissue (6 mm×6 mm) of a mouse was placed on the hydrated IEVs hydrogel. Then, the slide was flipped over to observe the adhesion ability of the IEVs hydrogel.

Results demonstrate that the IEVs hydrogel sponge and the PBS hydrogel sponge are compressible and injectable (FIG. 14B). When placed in the water, the hydrogel sponge quickly absorbs water and reforms into a hydrogel (FIG. 14C), and can firmly adhere to the heart, liver, spleen, lung, kidney or skin tissue of the mouse (FIG. 14D).

### 4. Blood Clotting Index (BCI)

30 μL of anticoagulated rat blood and CaCl₂) (0.2 mol·L⁻¹, 7 μL, Sigma, C5670) were respectively slowly dropwise added to the surface of an equivalent volume of sample or plate (negative control), and incubated at 37 °C for 5 min. 5 mL of PBS was carefully added without disturbing a blood clot. Then, the sample was gently shaken (37 °C, 120 rpm, 5 min). Absorbance of the solution at 540 nm was measured by using a microplate reader (SynergyH1, BioTek, U.S.A.). A gelatin sponge Surgifoam® and a collagen sponge Avitene™ were taken as positive controls.

BCI of different materials were calculated by the following formula:

BCI (%)=(ODₛₐₘₚₗₑ/OD_{blank control})×100%
where ODₛₐₘₚₗₑ is absorbance of the sample solution at 540 nm, and OD_{blank control} is absorbance of the negative control solution at 540 nm.

Experimental results demonstrate that BCI of the IEVs hydrogel sponge group is lower than that of the blank control group, the PBS hydrogel sponge, and the commercially available hemostasis product, namely, the gelatin sponge Surgifoam® and the collagen sponge Avitene™, indicating that the IEVs hydrogel sponge has excellent external hemostasis performance (FIG. 14E).

### 5. In Vitro IEVs Release Profile of IEVs Hydrogel Sponge

IEVs were labeled with a PKH26 red fluorescent cell linker kits (Sigma-Aldrich, U.S.A.) for synthesis of a PKH26-labeled IEVs hydrogel sponge. 60 mg of IEVs hydrogel sponge was immersed into 1 mL of PBS at 37 °C, supernatants were collected at preset time points (1 min, 3 min, 5 min, 7 min, 10 min, 1 h, 3 h, 7 h, 10 h, 24 h, respectively), and fluorescence intensity of each supernatant at 570 nm was measured by using a microplate reader.

Results demonstrate that the IEVs hydrogel sponge rapidly releases all IEVs within 1 h (FIG. 14F). The rapid release pattern indicates that IEVs can be quickly released to stop local bleeding and continuously released within 1 h to prevent secondary bleeding.

### 6. SEM

The IEVs hydrogel sponge and PBS hydrogel sponge were coated with gold for conductivity by using a sputter coater (MC1000, Hitachi, Japan) and observed by SEM (Quanta200, ThermoFisher, U.S.A.).

An SEM image shows that IEVs are visible on the surface of the IEVs hydrogel sponge (FIG. 14G), which ensures that the IEVs can come into direct contact with blood and participate in hemostasis as quickly as possible.

### Example 18 Hemostasis Effect of IEVs Hydrogel Sponge on Liver Bleeding in Rat

Preparation of IEVs hydrogel sponge and PBS hydrogel sponge was the same as that in Example 17.

250±20 g SD rats were randomly divided into the following groups according to the weight. The groups are: an IEVs hydrogel sponge group, a PBS hydrogel sponge group, a blank control group, a gelatin sponge Surgifoam® group, and a collagen sponge Avitene™ group, 4 rats per group. Experimental procedure: each rat was anesthetized, the abdomen was shaved, the largest lobe of the left liver was isolated, and a surgical area was cleaned with gauze to prevent exudate from interfering with experimental results. A liver tissue of the central part was taken with a drill to create a wound with a diameter of 8 mm and a depth of 5 mm. Then equal amounts of IEVs hydrogel sponge, PBS hydrogel sponge, gelatin sponge Surgifoam®, and collagen sponge Avitene™ were respectively placed at the bleeding point. For the blank control group, no material was placed. Filter paper was placed under the liver in advance to absorb blood. The blood loss was calculated by the following formula: blood loss=weight of filter paper after hemostasis-original weight of filter paper.

Results demonstrate that the hemostasis time of the IEVs hydrogel sponge group is shorter than that of the blank control group, the PBS hydrogel sponge group, and the commercially available hemostasis products, namely the gelatin sponge Surgifoam® and the collagen sponge Avitene™, and the blood loss is less, indicating that the IEVs hydrogel sponge has extremely excellent in vivo hemostasis performance (FIGS. 15A, B, and C).

### Example 19 Safety of IEVs Hydrogel Sponge after Liver Bleeding Surgery in Rat

After the liver bleeding surgery in the rats of Example 18, the skin was sutured and the abdominal cavity was closed. The rats were sacrificed 4 weeks later, and their livers were taken out and made into paraffin sections and frozen sections. Each paraffin section was stained with hematoxylin and eosin, and each frozen section was treated with CD3 and a mounting medium with 4′,6-diamidino-2-phenylindole (DAPI) (Abcam, ab104139).

Experimental results demonstrate that four weeks after surgery, the IEVs hydrogel sponge and other hemostasis materials cannot be completely degraded in the damaged liver (FIG. 15D), indicating that complete degradation of the material in the liver takes a longer period of time. It is noteworthy that in the liver of the untreated group (the blank control group), a large area of necrotic liver tissue was observed, which may be because the group without any treatment has the greatest blood loss. In addition, the IEVs hydrogel sponge and the untreated group show minimal inflammatory cell infiltration, indicating that the IEVs hydrogel sponge has good biocompatibility in vivo (FIG. 15 D, E). In contrast, after treatment with the gelatin sponge Surgifoam®, a large number of inflammatory cells infiltrate the damaged liver area. Therefore, the IEVs hydrogel sponge has excellent biocompatibility and shows promising clinical application prospects.

### Example 20 Hemostasis Effect of IEVs Hydrogel Sponge on Femoral Artery Bleeding in Rat

Preparation of IEVs hydrogel sponge and PBS hydrogel sponge was the same as that in Example 17.

250±20 g SD rats were randomly divided into the following groups according to the weight. The groups are: IEVs hydrogel sponge group, PBS hydrogel sponge group, blank control group, gelatin sponge Surgifoam® group, and collagen sponge Avitene™ group, 4 rats per group. Experimental procedure: each rat was anesthetized and shaved, the femoral artery was isolated, and a surgical area was cleaned with gauze to prevent exudate from interfering with experimental results. The femoral artery was clamped with an artery clamp, a hole (with a diameter of 0.5 mm) was poked in the femoral artery with a tip of a 1 mL needle, the artery clamp was loosened, the blood vessel was allowed to bleed freely for 10 s and then pressed with 3 pieces of pre-weighed gauze and a 200 g weight for 10 s. Then equal amounts of IEVs hydrogel sponge, PBS hydrogel sponge, the gelatin sponge Surgifoam®, and the collagen sponge Avitene™ were respectively placed on the bleeding point, and the bleeding point was covered with 5 pieces of pre-weighed gauze, and pressed with a 200 g weight. The hemostasis time was observed every 1 min. The blood loss was calculated by using the following formula: blood loss=weight of gauze after hemostasis−original weight of gauze.

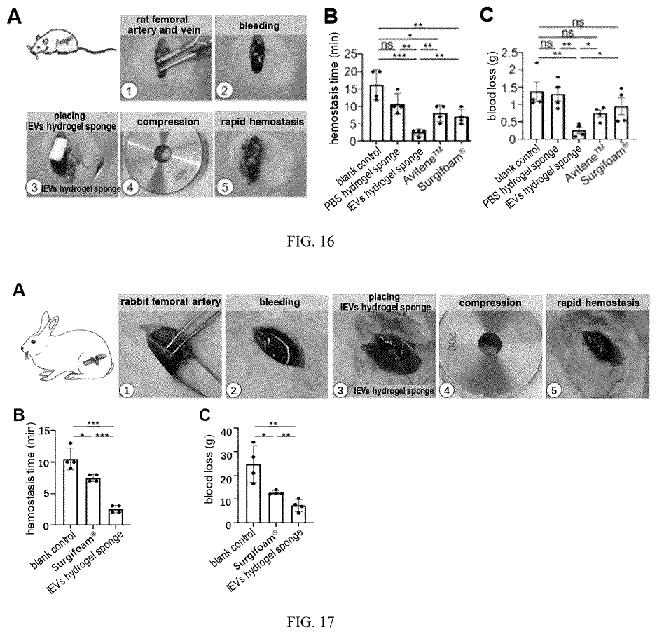

Results demonstrate that the hemostasis time of the IEVs hydrogel sponge group is shorter than that of the blank control group, the PBS hydrogel sponge, the gelatin sponge Surgifoam®, and the collagen sponge Avitene™, the blood loss is less, and differences are statistically significant (FIGS. 16A, B, and C), indicating that the IEVs hydrogel sponge has excellent in vivo hemostasis performance.

### Example 21 Hemostasis Effect of IEVs Hydrogel Sponge on Femoral Artery Bleeding in New Zealand White Rabbit

Preparation of IEVs hydrogel sponge and PBS hydrogel sponge was the same as that in Example 17.

2000±200 g New Zealand White Rabbits were randomly divided into the following five groups according to the weight. The groups are: an IEVs hydrogel sponge group, a PBS hydrogel sponge group, a blank control group, a gelatin group Surgifoam® group, and a collagen sponge Avitene™ group, 4 rabbits per group. Experimental procedure: each rabbit was anesthetized and shaved, the femoral artery was isolated, and a surgical area was cleaned with gauze to prevent exudate from interfering with experimental results. The femoral artery was clamped with an artery clamp, a hole (with a diameter of 0.5 mm) was poked in the femoral artery with a tip of a 1 mL needle, the artery clamp was loosened, and the blood vessel was allowed to bleed freely for 10 s and then pressed with 3 pieces of pre-weighed gauze and a 200 g weight for 10 s. Then, equal amounts of IEVs hydrogel sponge, PBS hydrogel sponge, the gelatin sponge Surgifoam®, and the collagen sponge Avitene™ were respectively placed on the bleeding point, and the bleeding point was covered with 5 pieces of pre-weighed gauze, and pressed with a 200 g weight. The hemostasis time was observed every 1 min. The blood loss was calculated by using the following formula: blood loss=weight of gauze after hemostasis-original weight of gauze.

Results demonstrate that the hemostasis time of the IEVs hydrogel sponge group is shorter than that of the blank control group, the PBS hydrogel sponge, the gelatin sponge Surgifoam®, and the collagen sponge Avitene™, the blood loss is less, and differences are statistically significant (FIGS. 17A, B, and C), indicating that the IEVs hydrogel sponge has excellent in vivo hemostasis performance.

1-15. (canceled)

16. A method for external procoagulation or external hemostasis in a subject, comprising administering a pharmaceutical composition comprising induced vesicles to the subject.

17. The method of claim 16, wherein the induced vesicles are tissue factor-positive induced vesicles.

18. The method of claim 16, wherein at least 30% of the induced vesicles in the pharmaceutical composition are tissue factor-positive induced vesicles.

19. The method of claim 16, wherein at least 60% of the induced vesicles in the pharmaceutical composition are tissue factor-positive induced vesicles.

20. The method of claim 16, wherein the pharmaceutical composition further comprises a tissue factor.

21. The method of claim 16, wherein the induced vesicles are derived from cells by inducing apoptosis of the cells.

22. The method of claim 21, wherein said inducing apoptosis is in the presence of staurosporine, by ultraviolet irradiation, by starvation, or by a heat stress.

23. The method of claim 21, wherein said inducing apoptosis is in the presence of staurosporine.

24. The method of claim 21, wherein the cells are stem cells.

25. The method of claim 21, wherein the cells are mesenchymal stem cells.

26. The method of claim 21, wherein the cells are mesenchymal stem cells derived from bone marrow, dental pulp, urine, oral cavity, adipose, placenta, umbilical cord, periosteum, tendon or peripheral blood.

27. The method of claim 21, wherein the cells are umbilical cord-derived mesenchymal stem cells.

28. The method of claim 16, wherein the pharmaceutical composition is selected from the group consisting of a freeze-dried powder injection, an injection, a nebulized inhalant, an oral preparation, and an external preparation.

29. The method of claim 16, wherein the pharmaceutical composition is an external preparation, and wherein the method comprises topically administering the external preparation to the subject.

30. The method of claim 29, wherein the external preparation is selected from the group consisting of a tincture, a cataplasm, paste, powder, an ointment, a plaster, a cream, a lotion, liniment, a spray, an aerosol, a film, a patch, gel, liquid, a sponge, and a gauze.

31. The method of claim 30, wherein the external preparation is a sponge.

32. The method of claim 16, wherein the pharmaceutical composition further comprises a pharmaceutically acceptable carrier.

33. The method of claim 32, wherein the pharmaceutically acceptable carrier comprises at least one member selected from the group consisting of a diluent, an excipient, a filler, a binder, a disintegrant, a surfactant, and a lubricant.

34. The method of claim 16, wherein the pharmaceutical composition further comprises a procoagulant or a hemostatic.

35. The method of claim 34, wherein the procoagulant or the hemostatic is at least one member selected from the group consisting of prothrombin, thrombin, fibrinogen, fibrin, cohesin, a coagulation factor, collagen, gelatin, vasopressin, a plasminogen activator inhibitor, a platelet activator, and a hemostatic synthetic peptide.
